(19) United States
(12) Patent Application Publication
(10) Pub. No.: US 2023/0201418 A1
(43) Pub. Date: Jun. 29, 2023

(54) SOFT TISSUE AUGMENTATION USING INJECTABLE, NEUTRAL PH SOLUBLE COLLAGEN-GLYCOSAMINOGLYCAN COMPOSITIONS

(71) Applicant: Shanghai Qisheng Biological Preparation Co., Ltd., Shanghai (CN)

(72) Inventors: Dale P. DeVore, Chelmsford, MA (US); Jiaxun ZHU, Shanghai (CN)

(21) Appl. No.: 18/089,811

(22) Filed: Dec. 28, 2022

Related U.S. Application Data
(60) Provisional application No. 63/294,118, filed on Dec. 28, 2021.

(51) Int. Cl. A61L 27/26; A61L 27/24; A61L 27/34; A61L 27/54

(52) U.S. Cl. CPC A61L 27/26; A61L 27/24; A61L 27/34; A61L 27/54; A61L 2300/402; A61L 2400/06; A61L 2430/34

(57) ABSTRACT

The present invention describes neutral pH soluble collagen-glycosaminoglycan compositions and methods for augmenting soft tissue defects using the compositions. Soft tissue defects include dermal wrinkles and dermal folds, dermal contour unevenness and laxity and subdermal volume deficiencies. The compositions may also be used for and promoting cellular growth and stimulating tissue regeneration.

### CROSS REFERENCE TO RELATED APPLICATION

This application claims the benefit of U.S. Provisional Application No. 63/294,118, filed Dec. 28, 2021, which is incorporated herein by reference in its entirety.

### FIELD OF INVENTION

The present invention describes methods for augmenting soft tissue using injectable, soluble collagen in neutral pH and natural glycosaminoglycan compositions. This invention was inspired by natural extracellular matrix compositions of collagen and macromolecular glycosaminoglycan (such as hyaluronic acid). Collagen provides excellent biocompatibility for cell adhesion and tissue integration. And macromolecular glycosaminoglycan (like hyaluronic acid or heparosan, especially cross-linked hyaluronic acid or heparosan) with good water retaining property and mechanical properties contributed to the longevity of soft tissue argumentation effect.

The compositions may also be used for stimulating tissue regeneration. The compositions are chemically treated to produce temperature stable viscous solutions at neutral pH. Upon injection into tissues, the solutions rapidly undergo gelation and polymerization to form fibrous collagen matrices containing cross-linked or uncross-linked macromolecular glycosaminoglycan.

### BACKGROUND

Collagen compositions have been utilized for more than 30 years to augment or smooth out soft tissue defects such as dermal wrinkles and dermal folds, to volumize furrows, or to correct dermal contour unevenness and laxity.

The collagen compositions utilized for soft tissue augmentation have been comprised of either reconstituted collagen fibrils prepared from solubilized collagen extracted from animal hides, reconstituted collagen fibrils prepared from soluble recombinant human collagen or intact collagen fibrils or fibers processed from human skin. In all cases the collagen composition has been composed of collagen fibrils/fibers or crosslinked collagen fibrils/fibers.

There are many references describing the application of collagen for soft tissue augmentation or for use as a dermal filler. Several key references are attached to this application. In addition there are many issued and pending patents referencing collagen for soft tissue augmentation. A list of these patents is also attached to this application.

Since soft tissues are primarily composed of collagen-based matrices, it makes sense to correct soft tissue defects with collagens or collagen-based compositions. There have been at least twelve FDA approved collagen products available for soft tissue augmentation in the U.S. since 1981. These products are generally called dermal fillers. However, at this time, most of the collagen-based fillers are no longer available in the U.S. market. They have been replaced by compositions that provide more durability including hyaluronic acid products, and products containing hydroxyapatite microbeads, poly-L-lactic acid particles, and polymethylmethacrylate microspheres.

There is still interest in having improved collagen-based compositions available for soft tissue augmentation because collagen serves as a scaffold capable of supporting cell attachment and cell proliferation, tissue integration in vivo through bioactive adhesion sites. The weakness of collagen-based composition of soft tissue augmentation is that collagen-based soft tissue filler generally undergo degradation and lost its augmentation effect in 3 to 6 months. Therefore, the compositions must exhibit increased durability.

Crosslinked macromolecular glycosaminoglycans like crosslinked hyaluronic acid are widely used for soft tissue augmentation because of its longevity and excellent safety profile. However, because macromolecular glycosaminoglycans lack cell adhesion, they are usually ‘inert’ to cell or tissue integration. (FIG. 9). Combining collagen and macromolecular glycosaminoglycan was a strategy to develop soft tissue scaffold with cellular growth promoting properties and long duration in tissue space reducing lines, folds, fine lines, wrinkles, or scars, or a combination thereof. Dr. Oded Shoseyov and his colleagues invented photoinitiated dermal fillers, hyaluronic acid-collagen double crosslinked dermal fillers (U.S. patent Ser. No. 17/052,216 assigned to Collagen Ltd). Light was applied to the surface of the epidermis superficial to induce polymerization of the combination including photoinitiator described in the patent.

Collagen is sensitive to temperature and ionic strength which drives spontaneous gel formation at proper temperature, under physiological conditions. The present invention describes methods for augmenting soft tissue using collagen-glycosaminoglycan compositions in the form of a viscous, biocompatible gel that can be easily injected through small needles (e.g., 27 gauge) and upon injection into tissues, rapidly undergoes gelation and fibril formation. The formed collagen-glycosaminoglycan matrix exhibits unique properties that prolong durability beyond that of any of the current injectable collagen fillers, and promoting cell ingrowth, tissue integration, healing or replacement due to degradation or injury of a collagen-comprising tissue beyond any of the current injectable hyaluronic acid products, and products containing hydroxyapatite microbeads, poly-L-lactic acid particles, and polymethylmethacrylate microspheres.

### SUMMARY OF INVENTION

The disclosure herein relates to an injectable soft tissue filler comprising derivatized collagen or in situ polymerizing collagen and glycosaminoglycan, form a cellular growth promoting scaffolds, as well as methods of using the soft tissue fillers in some instances, for soft tissue augmentation.

In one aspect of the present application, provided herein is a composition for soft tissue augmentation comprising: (i) neutral pH soluble collagen; and (ii) glycosaminoglycan; and (iii) optionally, other active ingredients, wherein the neutral pH soluble collagen was mixed with glycosaminoglycan.

In some embodiments, the neutral pH soluble collagen is selected from the group consisting of derivatized collagen or in situ polymerizing collagen, or a combination thereof. In some embodiments, the glycosaminoglycan is selected from the group consisting of crosslinked and/or non-crosslinked glycosaminoglycan.

In some embodiments, said other active ingredients is selected from the group consisting of:

(a) a plasma or a platelet-rich plasma or at least one growth factor comprises plasma or platelet-rich plasma, preferably in a concentration of 1%-50% by weight;

(b) cell free fat extract or at least one growth factor comprises cell free fat extract, preferably in a concentration of 0.1%-5% by weight;

(c) cell free stem cell extract or at least one growth factor comprises cell free stem cell extract, preferably in a concentration of 0.1%-5% by weight;

(d) Extracellular Vehicles (EVs), secreted by stem cells, preferably in a concentration of 0.1%-5% by weight;

(e) essential amnio acids or at least one essential amino acid, preferably in a concentration of 0.1%-5% by weight;

(f) polynucleotide(PN) and/or polydeoxyribonucleotide (PDRN) extracted from the sperm cells of Oncorhynchus mykiss (Salmon trout) or Oncorhynchus keta (Chum Salmon) with a molecular weight ranging from 50 to 1500 kDa, preferably in a concentration of from 0.1˜2% by weight;

(g) local anesthesia drugs such as lidocaine, procaine, preferably in a concentration of from 0.1% to 0.5% by weight;

(h) stabilizer or dissolution promotor, such as Methyl sulfonyl methane (MSM), preferably in a concentration of from 0.1% to 5% by weight; and

(i) any combinations thereof.

In some embodiments, the ratio of glycosaminoglycan to the neutral pH soluble collagen is between 10:1 to 1:10. In some embodiments, the concentration of glycosaminoglycan is in a range between 5 to 50 mg/ml.

In some embodiments, the source of collagen is selected from allogenetic tissue, mammal tissue (usually porcine, bovine, equine hides OR tendon) or marine species or axolotl hides derived matrix.

In some embodiments, the collagen is selected from full collagen or atelocollagen, or recombinant collagen or recombinant collagen peptides from microorganism, plants, insect cells or animal cells, or collagen mimic peptides.

In some embodiments, the derivatized collagen is derivatized with acetylation agents that alter the pKa of collagen and has one or more of the following features: (a) soluble at neutral pH (such as 6.5-7.5); (b) does not undergo fibrillogenesis at physiological pH; and/or (c) precipitates at acidic pH (such as 3.5-5.5, preferred 4.0˜5.0).

In some embodiments, the derivatized collagen is derivatized with one or more agents selected from the group consisting of glutaric anhydride, succinic anhydride, maleic anhydride, citric acid anhydride, oxalic acid anhydride and ethylenediamine tetraacetic anhydride.

In some embodiments, the neutral pH soluble collagen forms rapidly polymerizing collagen gels as described in U.S. Pat. No. 10,111,981B2.

In some embodiments, the rapidly polymerizing collagen gels comprises a neutralized solution comprising an acid soluble collagen, EDTA/EGTA and a polyol, and wherein the acid soluble collagen comprises collagen selected from the group consisting of Type I collagen, Type II collagen, Type III collagen and combinations thereof.

In some embodiments, the acid soluble collagen in a concentration between 5 and 70 mg/ml. In some embodiments, said EDTA is disodium EDTA; and/or said EGTA is disodium EGTA. In some embodiments, EDTA or EGTA is in a concentration between 10 and 50 mM. In some embodiments, said polyol is a sugar alcohol, such as D-mannitol. In some embodiments, polyol is in a concentration between 2.5% and 4% (w/v). In some embodiments, said rapidly polymerizing collagen gels further comprises a disaccharide, fructose, or combinations thereof. In some embodiments, said rapidly polymerizing collagen gel has an osmolality of 280-360 mmol/kg.

In some embodiments, the glycosaminoglycan is one or more selected from the group consisting of hyaluronic acid, heparosan, heparin, chondroitin sulfate, dermatan sulfate, keratan sulfate, and any combinations thereof.

In some embodiments, the glycosaminoglycan is derived from allogenetic tissue, mammal tissue or marine species; and/or is produced through microbial fermentation.

In some embodiments, the molecular weight of glycosaminoglycan before crosslinking is from 1000 Da˜10000000 Da.

In some embodiments, the crosslinker crosslinking Glycosaminoglycan are independently selected from 1,4-butanediol diglycidyl ether (BDDE), 1-[3-(Dimethylamino)propyl] ethylcarbodiimide methiodide (EDC), polyethyleneglycol diglycidyl ether (PEGDE), N,N′-dicyclohexylcarbodiimide (DCC), N,N′-dii sopropylcarbodiimide (DIC), Diepoxyoctane (DEO), Divinyl Sulfone (DVS), glutaraldehyde, or p-phenylene biscarbodiimide or 1,2,7,8-diepoxyoctane, or Polyethylene glycol (PEG), or oligomers rich in amino groups (such as poly-lysine or poly-arginine or γ-polyglutamic acid) or combinations thereof.

In some embodiments, hyaluronic acid is selected from oligo-hyaluronan, hyaluronic acid produced by microbial fermentation using Streptococcus species or Bacillus species, or allogenetic or animal tissues (including rooster combs, human umbilical cord, bovine synovial fluid or vitreous humor) derived hyaluronic acid.

In some aspect of the present application, provided herein is a method of preparing a composition comprising (i) neutral pH soluble collagen; (ii) glycosaminoglycan; and (iii) optionally, other active ingredients, said method comprises one or more step selected from:

(a) combining part (i) with part (ii), for example by adding part (ii) to part (i) by utilizing vacuum planetary mixer, to form an injectable homogeneous gel, preferably with a revolution speed of 200 rpm˜1,400 rpm and an autorotation speed of 100 rpm˜700 rpm, preferably with a mixing time of 10˜30 minutes with vacuum under sterile condition; or

(b) adding ethanol precipitated part (ii) to a salt or pH precipitate of part (i) and adding part (iii) (if present) and re-solubilizing the combination by dialysis or diafiltration or ultrafiltration process to form a homogeneous injectable gel; or

(c) combining part (i),part (ii) and part (iii) (if present), for example by sterile freeze-drying part (i) and part (ii) and part (iii), and re-solubilizing the mixture of lyophilized part (i),part (ii) and part (iii) and dialyzing the combination to neutral pH form a homogeneous injectable gel.

In some aspect of the present application, provided herein is a method for augmenting soft tissue or inducing a cellular growth promoting scaffold in a tissue space under an epidermis in a subject in need thereof, comprising administering the composition of claim 1 to a site in need of the augment or induction.

In some embodiments, the composition is injected into soft tissue to correct soft tissue deficiencies. In some embodiments, the composition is injected into dermis to correct soft tissue deficiencies including wrinkles, dermal folds, dermal laxity, unevenness, facial emaciation, fat atrophy, cheek depression, eye socket depression, or a combination thereof. In some embodiments, the composition is injected into tissues other than dermis, including cartilage, to correct tissue deficiencies.

In some embodiments, the composition is injectable through a 25˜30 gauge needle or cannula, such as a 25, 27 or 30 gauge needle or cannula.

### BRIEF DESCRIPTION OF THE DRAWINGS/FIGURES

In the following, aspects of the invention will be elucidated by means of examples, with reference to the drawings. The drawings are diagrammatic and may not be drawn to scale. The following drawings form part of the present specification and are included to further demonstrate certain aspects of the present invention. The invention may be better understood by reference to one or more of these drawings in combination with the detailed description of specific embodiments presented herein.

FIG. 1 shows In situ collagen polymerization in saline solution. FIG. 1A: Injection of in situ polymerizable collagen into saline; FIG. 1B: 60 seconds after injection of in situ polymerizable collagen in saline solution.

FIG. 2 shows a photograph of Collagen-HA composition (80% In situ polymerizing collagen+20% HA gel).

FIG. 3 shows a photograph of Collagen-HA composition (80% In situ polymerizing collagen+20% HA gel) polymerized in 2 minutes after injected into 37° buffer solution.

FIG. 4 shows a photograph of Collagen—crosslinked-HA composition (50% In situ polymerizing collagen+50% CXL-HA gel) polymerized in 2 minutes after injected into 37° buffer solution.

FIG. 5 shows a TEM image of polymerized collagen-crosslinked-HA composition.

FIG. 6 shows a TEM image of collagen fibrils of polymerized collagen-crosslinked-HA composition.

FIG. 7 shows H&E stain of collagen-crosslinked-HA composition implant in rabbit ear (10× magnification).

FIG. 8 shows H&E stain of collagen-crosslinked-HA composition implant in rabbit ear. Arrows showed cell ingrowth induced by the presence of collagen (20× magnification).

FIG. 9 shows H&E stain of Crosslinked-HA composition implant in rabbit ear. Very few cell ingrowth in the implantation (20× magnification).

FIG. 10 shows Injection force of derivatized collagen-heparosan-PRP composition measured by UTM.

### DETAILED DESCRIPTION OF THE INVENTION

Described herein is a method for augmenting soft tissues using a combination of a soluble collagen and glycosaminoglycan especially hyaluronic acid capable of rapid polymerization when in contact with or mixed with tissue fluids. This method of using this collagen-glycosaminoglycan composition may augmenting soft tissues, such as correcting skin contour defects, or for enhancing soft tissue regeneration.

The in situ polymerizing collagen is a clear, viscous, soluble collagen at neutral pH that upon interacting with tissue fluids, instantly forms a cohesive clear gel that rapidly undergoes fibril formation to form an opaque collagen matrix which was described in U.S. Ser. No. 10/111,981B2 and U.S. Ser. No. 11/235,089B2. Upon injection into tissue, such as dermal tissue, the in situ polymerizing collagen forms a fibrous mass that has been shown to retain volume for time periods beyond 6 months.

The base collagen used to prepare the in situ polymerizing collagen may be extracted from animal hides, such as bovine hide or porcine hide, or may be cell derived human collagen, or recombinant human collagen. It is preferred that the base collagen be available in acid solution. Any acid soluble, fibril forming collagen type may be used. However, Type I, Type II, Type III collagen or their combination are preferred to prepare the in situ polymerizing collagen.

Particularly preferred collagen compositions for use in the invention are described in DeVore & Eiferman (U.S. Pat. No. 5,492,135 assigned to Euclid Systems Corporation). These collagen compositions are initially soluble in form and, upon exposure to physiological fluids in vivo, undergo rapid polymerization. Such collagen solutions have been prepared at concentrations ranging from 10 mg/ml to over 70 mg/ml and at a pH ranging from 6.0-8.0.

In some embodiments of the invention, a neutralized, acid solubilized collagen, which remains in solution at physiological temperatures, is used to prepare in situ polymerizing collagen for soft tissue augmentation. These solutions must be extensively dialyzed against EDTA solutions and/or deionized water to reduce available cations and to prevent premature collagen fibrillogenesis. As the cations are removed, the pH of the collagen solution is increased to between about 6.8 and about 7.5 by adjusting the pH of the EDTA solution using 1N sodium hydroxide. The collagen preparation does not undergo typical fibrillogenesis in the absence of added unbound or free cations

In preferred embodiments, upon administration of the soluble collagen, the solution is converted to a gel or polymerized into a collagen fibrillar mass within 180 seconds, more preferably, within 120 seconds, most preferably, within 90 seconds. Preferably, the collagen-based solution is at a concentration of between 0.1-10%, more preferably, 0.5-7%, and most preferably between 2-5% collagen solids (w/v).

Glycosaminoglycan, also known as mucopolysaccharide, is a class of negatively-charged polysaccharide compounds. They are composed of repeating disaccharide units that are present in every mammalian tissue. Glycosaminoglycan is highly biological compatible.

Glycosaminoglycan, such as hyaluronic acid and heparosan now can be produced from microbial fermentation and are widely used as soft tissue augmentation and intraarticular viscosupplement. And the addition of glycosaminoglycan or crosslinked Glycosaminoglycan does not affect in situ polymerizing collagen polymerization property. Accordingly, it is an object of the invention to provide a method for using a neutralized, acid solubilized collagen-glycosaminoglycan solution suitable for use in soft tissue augmentation. When such compositions are injected into tissues, they quickly undergo gel formation and subsequent rapid fibrillogenesis when contacted with tissue fluids containing cationic constituents such as sodium chloride.

The composition has been injected into rabbit ear and examined histologically for biocompatibility. Results demonstrated the collagen-glycosaminoglycan composition implant has improved durability comparing to collagen based implant with little to no reduction in original injection volume.

Another neutral pH soluble collagen solution is derivatized collagen in which the isoelectric point of collagen was altered from around 7 to 4 by the acylation of collagen.

Acylation reactions have been used to derivatize soluble and insoluble collagen and have been described by DeVore, et. al. in a series of patents (U.S. Pat. Nos. 4,713,446; 4,851,513; 4,969,912; 5,067,961; 5,104,957; 5,201,764; 5,219,895; 5,332,809; 5,354,336; 5,476,515; 5,480,427; 5,631,243; 6,161,544 and 17,744,428). However, none of these patents describe the use of chemically derivatized collagen combined with glycosaminoglycan substances, such as to treat soft tissue deficiencies or defects.

In the present invention, the chemically modified collagen-glycosaminoglycan compositions can be injected into superficial dermis, mid-dermis, or deep dermis to correct contour defects in facial skin or such compositions can be injected into the loose connective tissue surrounding lip muscle or into the body of the lip to enhance lip appearance. The collagen compositions are injectable through a 30 gauge needle. The material remains colorless and provides a long-lasting clinical effect. The collagen compositions can be prepackaged in ready-to-use syringes containing materials exhibiting several different degrees of durability

### Definitions

By “collagen” is meant all forms of collagen including those which have been processed or modified. The collagen may be of human or animal origin or may be produced using recombinant techniques. The present invention can use these and other typed of collagen including natural collagen and various collagen derivatives.

By “tissue” is meant an aggregation of similarly specialized cells in an organism, preferably, mammalian, and, most preferably, human, where the cells are exposed to the organism's extracellular fluid, and are united in performance of a function within an organism.

By “in situ polymerization” is meant formation of a collagen gel and subsequently a collagen fibrous mass, upon injection of soluble collagen into tissue.

The present invention provides a number of advantages. For example, the collagen compositions described herein are biocompatible, biodegradable, and stable in solution at neutral pH. The ability to chemically manipulate the collagen to form a neutral stable solution allows for injectable administration through a fine gauge needle (e.g., a 30 or 31 gauge needle). In addition to the ease of application, injectable delivery of the collagen solution allows access to the administration site while minimizing invasive injury to surrounding tissues. The density of the collagen solution is sufficient to fill a soft tissue defect or other specific delivery site and remain in place until gelation and fibril formation occurs, and maintenance of soft tissue augmentation for at least 6 months.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Preparation of In Situ Polymerizing Collagen Solution

The in situ polymerizing collagen was prepared using methods described previously by DeVore and Eiferman (U.S. Pat. No. 5,492,135; assigned to Euclid Systems Corporation). Pure soluble Type I collagen was purchased from Advanced BioMatrix, Inc. Sodium chloride was added to the soluble, pepsin-digested collagen solution (3 mg/mL) to a concentration of 0.8M to precipitate collagen. The white, opaque precipitate was recovered by centrifugation for 30 minutes at 3500 RPM and concentrated to approximately 50 mg/mL by placement on filter paper. The concentrated collagen precipitate was placed in dialysis tubing with a MW cut off of 100,000 and dialyzed against 0.1N HCl for 16-18 hours. The resulting clear, viscous, redissolved collagen concentrate was then dialyzed against 0.035M EDTA (ethylenediaminetetraacetic acid, disodium salt dihydate, SigmaUltra˜99%). Dialysis was continued for 5 days with daily adjustment of pH from the starting pH of 4.5 to a final pH of 7.5. The final clear and viscous collagen concentrate was collected and centrifuged to remove air bubbles. The final clear, viscous collagen exhibited a pH of 7.4 and did not undergo fibril formation at room temperature. Collagen fibrillogenesis was not triggered by pH or temperature.

Evaluation of gelation and fibril formation.

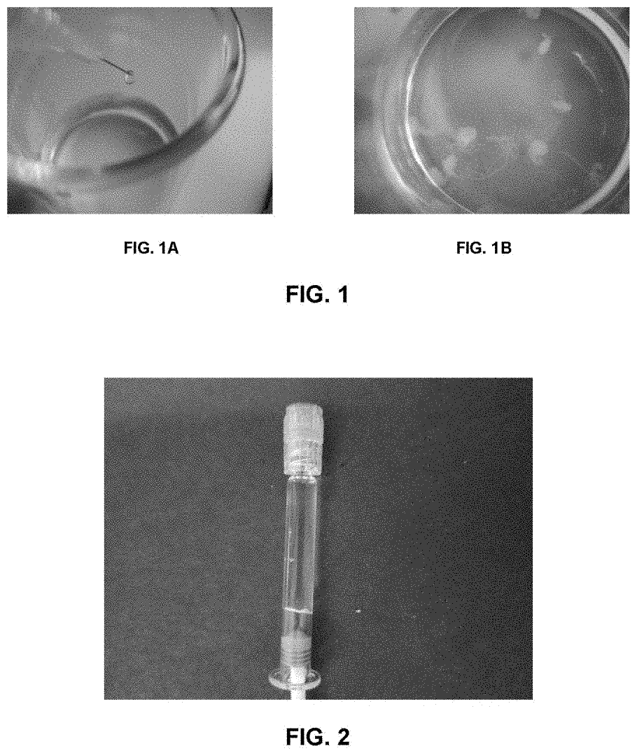

Aliquots of the in situ polymerizing collagen were injected into 0.8M sodium chloride at 37° C. and observed for the appearance of gel and fibrous collagen. As shown in FIG. 1 the clear viscous collagen solution formed a white, opaque collagen matrix in less than 60 seconds.

### Example 2. Evaluation of Gelation and Fibril Formation of In Situ Polymerizing Collagen-Hyaluronic Acid Composition

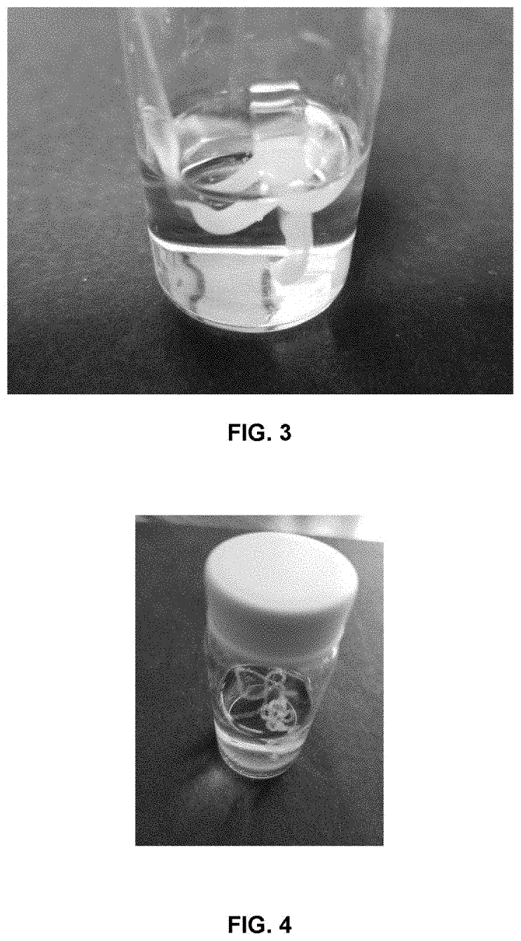

The in situ polymerizing collagen-hyaluronic acid composition was made by directly mixing 24 mg/mL in situ polymerizing collagen with 12 mg/mL hyaluronic acid PBS solution at 80:20 by weight and centrifuging at 6000 rpm to remove bubbles (FIG. 2). And the in situ polymerizing collagen-hyaluronic acid composition was injected into 0.8M sodium chloride at 37° C. and observed for the appearance of gel. As shown in FIG. 3 the clear viscous solution formed a white, opaque matrix in less than 120 seconds.

### Example 3. Evaluation of Gelation and Fibril Formation of In Situ Polymerizing Collagen-Crosslinked Hyaluronic Acid Composition

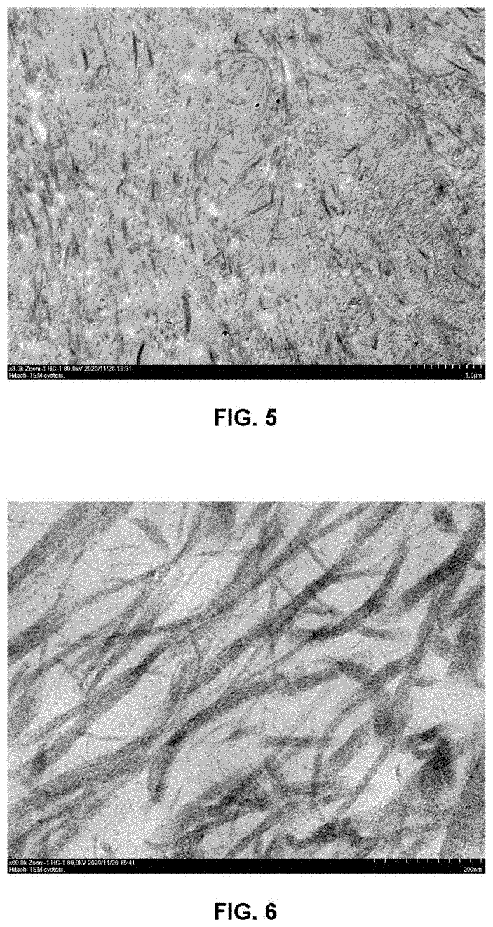

The in situ polymerizing collagen-hyaluronic acid composition was made by directly mixing 24 mg/mL in situ polymerizing collagen with commercial crosslinked hyaluronic acid (Restylane®) at 50:50 by weight and centrifuging at 6000 rpm to remove bubbles. And the in situ polymerizing collagen-crosslinked hyaluronic acid composition was injected into 0.8M sodium chloride at 37° C. and observed for the appearance of gel. As shown in FIG. 4 the clear viscous solution formed a slightly opaque gel. Transmission electron microscopy images (FIGS. 5 & 6) were taken for the gel, and collagen fibril structures were observed.

### Example 4. Evaluation of Biological Compatibility and Cell/Tissue Integration of In Situ Polymerizing Collagen-Crosslinked Hyaluronic Acid Composition in Rabbit Ear

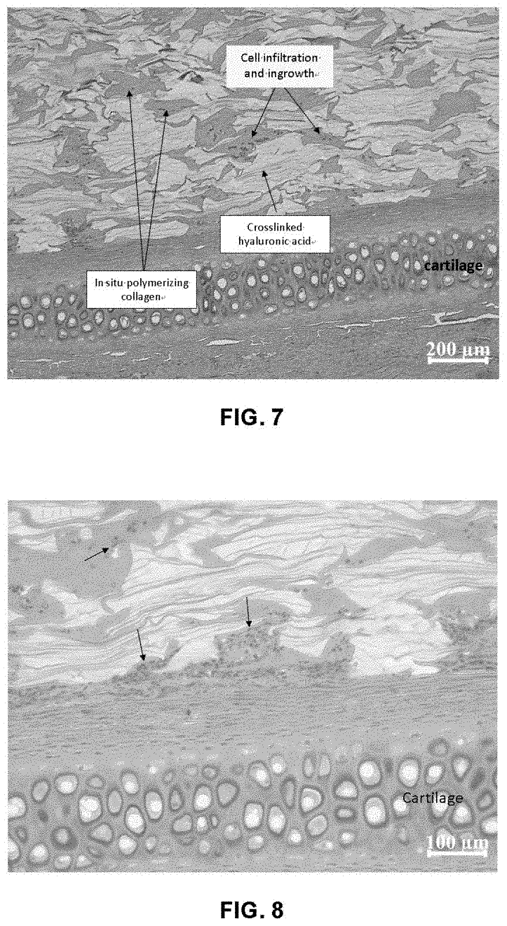

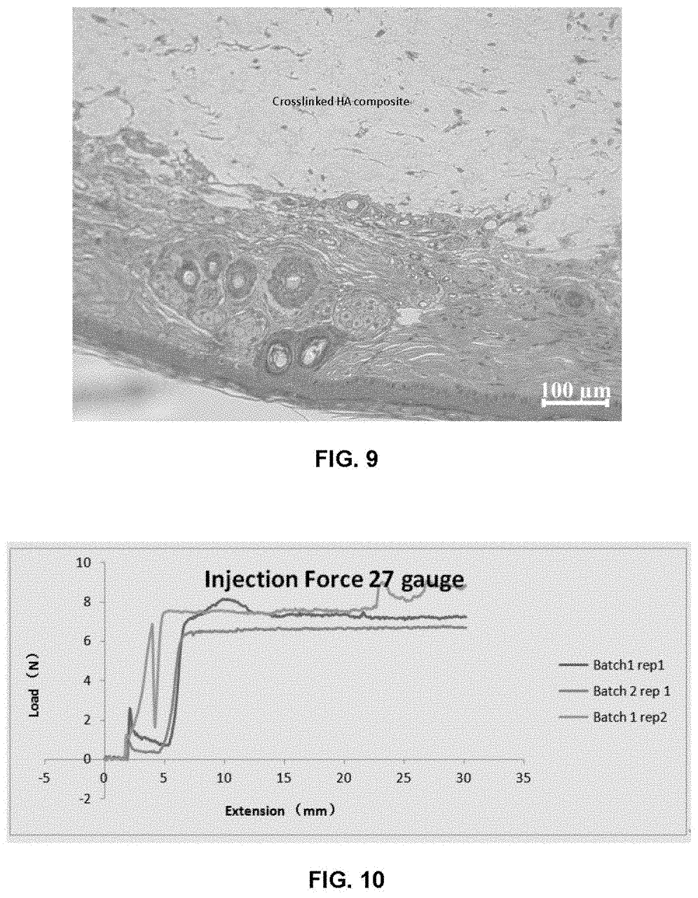

Five New Zealand rabbits were housed following the protocols from the guidelines for the use of laboratory animals. Up to 0.25 ml of in situ polymerizing collagen-crosslinked hyaluronic acid composition was injected via 27 or 25 gauge needle. After 4 weeks, two rabbits were euthanized followed by harvesting of the entire ear. Each ear was placed in formalin for histology. The implants were cut in the cross-section of maximum height and tissue block. Hematoxylin and eosin (H&E) stained slides at 10× (FIG. 7) and 20× magnification (FIG. 8) were examined to evaluate biological compatibility of in situ polymerizing collagen-crosslinked hyaluronic acid composition. Cellular infiltration induced by the collagen composition was observed by injection of the in situ polymerizing collagen-crosslinked hyaluronic acid composition, whereas the injection of Crosslinked-HA composition shows very few cell ingrowth in the implantation (FIG. 10).

The rabbit ear thickness, total thickness, implantation length and width in the rest three rabbits were measured three times by the same person with a vernier caliper right after implantation, 1 week, 4 weeks, 8 weeks and 12 weeks after implantation. The mean of the three measurements were used. The height of the implantation was calculated through total thickness of the implantation and rabbit ear minus rabbit ear thickness. And the volume was calculated using the ellipsoid volume formula. With cell ingrowth induced by collagen, the implantation of collagen-crosslinked hyaluronic acid showed a better augmentation effect and long duration comparing to crosslinked hyaluronic acid alone.

TABLE 1
Estimate height and volume of in situ polymerizing collagen-crosslinked hyaluronic
acid and crosslinked hyaluronic acid implantation in rabbit ears.

| | Estimated Height of | | | | | Estimated Volume of | | | | |
|---|---|---|---|---|---|---|---|---|---|---|
| | the implantation (mm) | | | | | the implantation (mm³) | | | | |
| | 0 | 1 week | 4 weeks | 8 weeks | 12 weeks | 0 | 1 week | 4 weeks | 8 weeks | 12 weeks |
| | rabbit 1 | | | | | | | | | |
| Collagen + | 4.16 | 2.45 | 2.68 | 1.96 | 2.73 | 347.20 | 157.86 | 252.36 | 203.06 | 217.89 |
| HA | | | | | | | | | | |
| HA | 1.67 | 1.32 | 1.43 | 1.27 | 0.87 | 125.60 | 96.27 | 119.34 | 96.75 | 66.65 |
| | rabbit 2 | | | | | | | | | |
| Collagen + | 3.98 | 2.48 | 3.63 | 4.75 | 3.19 | 258.59 | 182.82 | 381.71 | 624.39 | 307.72 |
| HA | | | | | | | | | | |
| HA | 2.88 | 2.36 | 2.18 | 1.43 | 1.98 | 200.07 | 231.03 | 221.15 | 151.61 | 222.69 |
| | rabbit 3 | | | | | | | | | |
| Collagen + | 3.37 | 2.41 | 3.57 | 2.88 | 4.09 | 187.71 | 148.67 | 239.93 | 257.56 | 746.17 |
| HA | | | | | | | | | | |
| HA | 2.06 | 2.35 | 2.24 | 1.82 | 2.12 | 114.17 | 177.18 | 162.06 | 118.77 | 196.99 |

### Example 5. Preparation of Derivatized Collagen-Heparosan-Platelet Rich Plasma (PRP) Composition

200 mL of 3 mg/mL purified, soluble collagen (Porcogen, Lot #531131080) was filtered through 0.45 μm and 0.2 μm cartridge filters. The filtered collagen was place in a 500 mL beaker and adjusted to a pH of 9.0 using 10N and 1N NaOH. After stirring for 5 minutes at room temperature, pulverized glutaric anhydride powder (Sigma, >95%) was slowly added to the stirring collagen solution at a concentration equal to 10% of the collagen (60 mg). The pH of the collagen solution was maintained at pH 9.0 by addition of drops of 10N NaOH. The glutaric anhydride reaction continued for 15 minutes at which point drops of 6N HCl and 1N HCl were added to reduce the pH to approximately 4.5 to precipitate the derivatized collagen. The derivatized collagen was then placed in 50 mL centrifuge tubes and centrifuged at 3,500-5,000 rpm to precipitate the derivatized collagen. The recovered precipitate was then solubilized by adjusting the pH to 7.2 by adding drops of 10 N NaOH and 1N NaOH. The pH was monitored as NaOH was mixed with the derivatized collagen pellet. The neutralized, clear and transparent collagen gel was then placed in 50 mL centrifuge tubes and centrifuged to remove air bubbles.

The derivatized collagen was diluted to 2 mg/mL and lyophilized under 0° C. for 48 hours. 0.1 grams of sodium heparosan powder (HTL Biotechnology, MW: 1800 kDa˜2400 kDa) was added to 0.7 grams of lyophilized collagen sponge. And 15 mL sterile PBS was used to re-solubilize collagen-heparosan mixture and the mixture was shaken at 50 rpm under 10° C. for 72 hours. The neutralized, clear and transparent collagen-heparosan gel was placed in the tube and centrifuged to remove air bubbles. Derivatized collagen-heparosan-PRP composition was produced by adding 5 ml PRP to the gel and homogeneously mixing by shaker at 50 rpm under 10° C. for 2 hours. PRP was prepared using human peripheral blood with Regenlab kit. Derivatized collagen-heparosan-PRP gel was loaded into 1 mL BD glass syringes and centrifuged at 3000 rpm for 5 min to remove bubbles.

27 gauge needle was attached to the syringe and the injection force of the composite through 27 gauge needle was evaluated by measuring compression force applied to syringe plug by universal testing machine (UTM). The injection force of derivatized collagen-heparosan-PRP gel was lower than 10N (FIG. 10).

Although the present invention has been described with reference to exemplary embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

### REFERENCES

Relevant Technical Publications
1. Denton, A B and Shoman, N. Chapter 13-“Review of Collagen Fillers” in Office-based

Cosmetic Procedures and Techniques. Cambridge University Press. Pp 59-64. 2010
2. Baumann, L, Blyumin, M, and Saghari, S. Chapter 23-“Dermal Fillers”, in Cosmetic

Dermatology-Principles and Practice. McGraw Hill pp191-211. 2009

Patents Referencing Collagens for Soft Tissue Augmentation

| Citing | Filing | Issue | Original | |
|---|---|---|---|---|
| Patent | date | date | Assignee | Title |
| U.S. Pat. No. | Oct. 9, 1979 | Sep. 22, 1981 | Merck Patent | Medicinally useful, shaped mass of |
| 4,291,013 | | | Gesellschaft Mit | collagen resorbable in the body |
| | | | Beschrankter | |
| | | | Haftung | |
| U.S. Pat. No. | Jun. 15, 1981 | Aug. 31, 1982 | Merck Patent | Medicinally useful, shaped mass of |
| 4,347,234 | | | Gesellschaft mit | collagen resorbable in the body |
| | | | beschrankter | |
| | | | Haftung | |
| U.S. Pat. No. | Jan. 11, 1982 | Jan. 3, 1984 | Collagen | Collagen implant material and |
| 4,424,208 | | | Corporation | method for augmenting soft tissue |
| U.S. Pat. No. | Sep. 15, 1983 | Dec. 18, 1984 | | Non-antigenic collagen and articles |
| 4,488,911 | | | | of manufacture |
| U.S. Pat. No. | Oct. 22, 1984 | Apr. 15, 1986 | Collagen | Injectable cross-linked collagen |
| 4,582,640 | | | Corporation | implant material |
| U.S. Pat. No. | Jul. 25, 1984 | Jun. 3, 1986 | Koken Co., Ltd. | Aqueous atelocollagen solution and |
| 4,592,864 | | | | method of preparing same |
| U.S. Pat. No. | Mar. 22, 1985 | Feb. 10, 1987 | Collagen | Mechanically sheared collagen |
| 4,642,117 | | | Corporation | implant material and method |
| U.S. Pat. No. | Aug. 6, 1986 | Dec. 15, 1987 | Minnesota Mining | Viscoelastic collagen solution for |
| 4,713,446 | | | and Manufacturing | ophthalmic use and method of |
| | | | Company | preparation |
| U.S. Pat. No. | Jun. 25, 1986 | Feb. 7, 1989 | Collagen | Injectable implant composition |
| 4,803,075 | | | Corporation | having improved intrudability |
| U.S. Pat. No. | Oct. 5, 1987 | Jul. 25, 1989 | Minnesota Mining | Viscoelastic collagen solution for |
| 4,851,513 | | | and Manufacturing | opthalmic use and method of |
| | | | Company | preparation |
| U.S. Pat. No. | Oct. 28, 1988 | Nov. 28, 1989 | Minnesota Mining | Modified collagen compound and |
| 4,883,864 | | | and Manufacturing | method of preparation |
| | | | Company | |
| U.S. Pat. No. | Feb. 18, 1988 | Nov. 13, 1990 | Autogenesis | Human collagen processing and |
| 4,969,912 | | | Technologies | autoimplant use |
| U.S. Pat. No. | Sep. 16, 1988 | Feb. 12, 1991 | Collagen | Method of making molds with |
| 4,992,226 | | | Corporation | xenogeneic collagen/mineral |
| | | | | preparations for bone repair |
| U.S. Pat. No. | May 7, 1990 | Apr. 14, 1992 | | Viscoelastic collagen gel for |
| 5,103,840 | | | | ophthalmic surgery |
| U.S. Pat. No. | Feb. 28, 1990 | Apr. 14, 1992 | Autogenesis | Biologically compatible collagenous |
| 5,104,957 | | | Technologies, Inc. | reaction product and articles useful |
| | | | | as medical implants produced |
| | | | | therefrom |
| U.S. Pat. No. | Nov. 14, 1989 | Nov. 10, 1992 | Collagen | Collagen-polymer conjugates |
| 5,162,430 | | | Corporation | |
| U.S. Pat. No. | Jan. 22, 1992 | Apr. 13, 1993 | Autogenesis | Biologically compatible collagenous |
| 5,201,764 | | | Technologies, Inc. | reaction product and articles useful |
| | | | | as medical implants produced |
| | | | | therefrom |
| U.S. Pat. No. | Dec. 2, 1992 | Mar. 8, 1994 | Collagen | Collagen-polymer tubes for use in |
| 5,292,802 | | | Corporation | vascular surgery |
| U.S. Pat. No. | Nov. 6, 1992 | Apr. 19, 1994 | Johnson Medical | Injection syringe |
| 5,304,147 | | | Development Corp. | |
| U.S. Pat. No. | Aug. 23, 1993 | Apr. 26, 1994 | Collagen | Method of augmenting tissue with |
| 5,306,500 | | | Corporation | collagen-polymer conjugates |
| U.S. Pat. No. | Oct. 28, 1991 | Jun. 28, 1994 | Atrix Laboratories, | Biodegradable polymer |
| 5,324,519 | | | Inc. | composition |
| U.S. Pat. No. | Jul. 2, 1992 | Jun. 28, 1994 | Collagen | Biologically inert, biocompatible- |
| 5,324,775 | | | Corporation | polymer conjugates |
| U.S. Pat. No. | Jul. 30, 1992 | Jul. 12, 1994 | Collagen | Collagen-polymer conjugates |
| 5,328,955 | | | Corporation | |
| U.S. Pat. No. | Feb. 10, 1993 | Nov. 22, 1994 | Collagen | Device for treating fine superficial |
| 5,366,498 | | | Corporation | facial lines |
| U.S. Pat. No. | Jan. 5, 1994 | Dec. 27, 1994 | Collagen | Method of augmenting tissue using |
| 5,376,375 | | | Corporation | collagen-polymer conjugates |
| U.S. Pat. No. | Nov. 2, 1992 | Jan. 24, 1995 | Collagen | Method for treating fine superficial |
| 5,383,930 | | | Corporation | facial lines |
| U.S. Pat. No. | Feb. 17, 1994 | May 9, 1995 | Collagen | Collagen-polymer conjugates |
| 5,413,791 | | | Corporation | |
| U.S. Pat. No. | Apr. 19, 1994 | Jun. 27, 1995 | Collagen | High concentration homogenized |
| 5,428,024 | | | Corporation | collagen compositions |
| U.S. Pat. No. | Jan. 7, 1991 | Jul. 25, 1995 | Pasteur Merieux | New preparation of placenta |
| 5,436,135 | | | Serums et Vaccins | collagen, their extraction method |
| | | | Imedex | and their applications |
| U.S. Pat. No. | Jan. 5, 1995 | Aug. 29, 1995 | Collagen | Collagen-polymer conjugates |
| 5,446,091 | | | Corporation | containing an ether linkage |
| U.S. Pat. No. | May 2, 1994 | Dec. 12, 1995 | Collagen | Collagen-synthetic polymer |
| 5,475,052 | | | Corporation | matrices prepared using a multiple |
| | | | | step reaction |
| U.S. Pat. No. | Nov. 24, 1993 | Dec. 19, 1995 | Autogenesis | Method of making intraocular |
| 5,476,515 | | | Technologies, Inc. | lenses with injectable collagen- |
| | | | | based compositions |
| U.S. Pat. No. | Jul. 19, 1994 | Jan. 2, 1996 | Autogenesus | Biologically compatible collagenous |
| 5,480,427 | | | Technologies, Inc. | reaction product and articles useful |
| | | | | as medical implants produced |
| | | | | therefrom |
| U.S. Pat. No. | Sep. 9, 1992 | Feb. 20, 1996 | CoIlagenesis, Inc. | Collagen modulators for use in |
| 5,492,135 | | | | photoablation excimer laser |
| | | | | keratectomy |
| U.S. Pat. No. | Nov. 3, 1993 | Apr. 23, 1996 | Collagen | Glycosaminoglycan-synthetic |
| 5,510,418 | | | Corporation | polymer conjugates |
| U.S. Pat. No. | Jun. 7, 1995 | Aug. 27, 1996 | Collagen | Polymer conjugates ophthalmic |
| 5,550,188 | | | Corporation | devices comprising collagen- |
| | | | | polymer conjugates |
| U.S. Pat. No. | Nov. 3, 1993 | Oct. 15, 1996 | Collagen | Clear, chemically modified col- |
| 5,565,519 | | | Corporation | lagen-synthetic polymer |
| | | | | conjugates for ophthalmic |
| | | | | applications |
| U.S. Pat. No. | Jul. 28, 1995 | Jan. 7, 1997 | Isolagen | Use of autologous dermal |
| 5,591,444 | | | Technologies, Inc. | fibroblasts for the repair of skin and |
| | | | | soft tissue defects |
| U.S. Pat. No. | Jun. 6, 1996 | Aug. 26, 1997 | Isolagen | Use of autologous dermal |
| 5,660,850 | | | Technologies, Inc. | fibroblasts for the repair of skin and |
| | | | | soft tissue defects |
| U.S. Pat. No. | Jun. 6, 1996 | Sep. 9, 1997 | Isolagen | Autologous dermal fibroblasts for |
| 5,665,372 | | | Technologies, Inc. | the repair of skin and soft tissue |
| | | | | defects |
| U.S. Pat. No. | Jan. 8, 1997 | Sep. 1, 1998 | Collagen | Collagen-synthetic polymer |
| 5,800,541 | | | Corporation | matrices prepared using a multiple |
| | | | | step reaction |
| U.S. Pat. No. | Sep. 29, 1995 | Sep. 15, 1998 | Collagen | Collagen-based injectable drug |
| 5,807,581 | | | Corporation | delivery system and its use |
| U.S. Pat. No. | Nov. 8, 1995 | Oct. 20, 1998 | Collagen | Apparatus and method of mixing |
| 5,823,671 | | | Corporation | materials in a sterile environment |
| U.S. Pat. No. | Jun. 6, 1995 | Nov. 3, 1998 | Advanced Tissue | Methods for production of a |
| 5,830,708 | | | Sciences, Inc. | naturally secreted extracellular |
| | | | | matrix |
| U.S. Pat. No. | Jan. 3, 1997 | Nov. 24, 1998 | AutoImmune, Inc. | Method for preparation of type II |
| 5,840,848 | | | | collagen |
| U.S. Pat. No. | Sep. 8, 1997 | Jan. 12, 1999 | Isolagen | Use of autologous undifferentiated |
| 5,858,390 | | | Technologies, Inc. | mesenchymal cells for the repair of |
| | | | | skin and soft tissue defects |
| U.S. Pat. No. | Feb. 16, 1996 | Jan. 19, 1999 | Collagenesis, Inc. | Collagen modulators for use in |
| 5,861,486 | | | | photoablation eximer laser |
| | | | | keratectomy |
| U.S. Pat. No. | Dec. 18, 1996 | Feb. 23, 1999 | Cohesion | Crosslinked polymer compositions |
| 5,874,500 | | | Technologies, Inc. | and methods for their use |
| U.S. Pat. No. | Jan. 13, 1999 | Apr. 18, 2000 | Cohesion | Crosslinked polymer compositions |
| 6,051,648 | | | Technologies, Inc. | and methods for their use |
| U.S. Pat. No. | Jun. 26, 1997 | Jun. 6, 2000 | Atrix Laboratories, | Method and composition for |
| 6,071,530 | | | Inc. | treating a bone tissue defect |
| U.S. Pat. No. | Jun. 7, 1995 | Oct. 10, 2000 | Reprogenesis, Inc. | Injectable hydrogel compositions |
| 6,129,761 | | | | |
| U.S. Pat. No. | Apr. 30, 1999 | Dec. 26, 2000 | Cohesion | Method of using crosslinked |
| 6,166,130 | | | Technologies, Inc. | polymer compositions in tissue |
| | | | | treatment applications |
| U.S. Pat. No. | Oct. 29, 1998 | Sep. 4, 2001 | Advanced Tissue | Compositions and methods for |
| 6,284,284 | | | Sciences, Inc. | production and use of an injectable |
| | | | | naturally secreted extracellular |
| | | | | matrix |
| U.S. Pat. No. | Dec. 8, 2000 | Nov. 27, 2001 | Cohesion | Method of making crosslinked |
| 6,323,278 | | | Technologies, Inc. | polymer matrices in tissue |
| | | | | treatment applications |
| U.S. Pat. No. | Oct. 28, 1997 | Jan. 8, 2002 | BioScience | Method and process for the |
| 6,337,389 | | | Consultants, L.L.C. | production of collagen preparations |
| | | | | from invertebrate marine animals |
| | | | | and compositions thereof |
| U.S. Pat. No. | Jun. 15, 2001 | Oct. 1, 2002 | Cohesion | Compositions and systems for |
| 6,458,889 | | | Technologies, Inc. | forming crosslinked biomaterials |
| | | | | and associated methods of |
| | | | | preparation and use |
| U.S. Pat. No. | Aug. 17, 2001 | Mar. 18, 2003 | Cohesion | Cross-linked polymer compositions |
| 6,534,591 | | | Technologies, Inc. | and methods for their use |
| U.S. Pat. No. | Apr. 9, 2001 | Jan. 27, 2004 | Colbar R&D Ltd. | Cross-linked collagen matrices and |
| 6,682,760 | | | | methods for their preparation |
| U.S. Pat. No. | Sep. 30, 2002 | Dec. 21, 2004 | Cohesion | Methods for tissue repair using |
| 6,833,408 | | | Technologies, Inc. | adhesive materials |
| U.S. Pat. No. | Jan. 27, 2004 | Jun. 28, 2005 | Cohesion | Composition for administration of a |
| 6,911,496 | | | Technologies, Inc. | biologically active compound |
| U.S. Pat. No. | Nov. 28, 2001 | Jul. 12, 2005 | Bioscience | Method and process for the |
| 6,916,910 | | | Consultants | production of collagen preparations |
| | | | | from invertebrate marine animals |
| | | | | and compositions thereof |
| U.S. Pat. No. | May 28, 2003 | Apr. 11, 2006 | Organogenesis, Inc. | Process of making bioengineered |
| 7,025,916 | | | | collagen fibrils |
| U.S. Pat. No. | Nov. 26, 2003 | Jun. 20, 2006 | CrossCart, Inc. | Substantially non-immunogenic |
| 7,064,187 | | | | injectable collagen |
| U.S. Pat. No. | Sep. 16, 2004 | Jul. 17, 2007 | Evera Medical | Systems and devices for soft tissue |
| 7,244,270 | | | | augmentation |
| U.S. Pat. No. | Aug. 8, 2000 | Aug. 19, 2008 | Isolagen | Aug.mentation and repair of vocal |
| 7,412,978 | | | Technologies, Inc. | cord tissue defects |
| U.S. Pat. No. | Aug. 12, 2003 | Aug. 19, 2008 | Incept LLC | Composite hydrogel drug delivery |
| 7,413,752 | | | | systems |
| U.S. Pat. No. | Apr. 28, 2006 | Sep. 29, 2009 | Crosscart, Inc. | Substantially non-immunogenic |
| 7,595,377 | | | | injectable collagen |
| U.S. Pat. No. | Mar. 8, 2004 | Oct. 5, 2010 | Massachusetts | Injectable composition containing |
| 7,807,150 | | | Institute of | crosslinkable material and cells for |
| | | | Technology | forming animal tissue |
| | | | Children's Medical | |
| | | | Center Corporation | |
| U.S. Pat. No. | Jan. 31, 2006 | Feb. 8, 2011 | AngioDevice | Compositions and systems for |
| 7,883,693 | | | International GmbH | forming crosslinked biomaterials |
| | | | | and methods of preparation of use |
| U.S. Pat. No. | Mar. 6, 2009 | Apr. 26, 2011 | aap Biomaterials | Process for the production of |
| 7,932,354 | | | GmbH | collagen material |
| U.S. Pat. No. | Apr. 28, 2005 | Nov. 29, 2011 | AngioDevice | Compositions and systems for |
| 8,067,031 | | | International GmbH | forming crosslinked biomaterials |
| | | | | and associated methods of |
| | | | | preparation and use |
| U.S. Pat. No. | Sep. 21, 2007 | Dec. 27, 2011 | Purdue Research | Collagen preparation and method |
| 8,084,055 | | | Foundation | of isolation |
| U.S. Pat. No. | Dec. 22, 2003 | Feb. 28, 2012 | Anika Therapeutics, | Crosslinked hyaluronic acid |
| 8,124,120 | | | Inc. | compositions for tissue |
| | | | | augmentation |

1. A composition for soft tissue augmentation comprising:
i) neutral pH soluble collagen; and
ii) glycosaminoglycan; and
iii) optionally, other active ingredients,
wherein the neutral pH soluble collagen was mixed with glycosaminoglycan.

2. The composition of claim 1, wherein
i) the neutral pH soluble collagen is selected from the group consisting of derivatized collagen or in situ polymerizing collagen, or a combination thereof; and/or
ii) the glycosaminoglycan is selected from the group consisting of crosslinked and/or non-crosslinked glycosaminoglycan; and/or
iii) said other active ingredients is selected from the group consisting of
(a) a plasma or a platelet-rich plasma or at least one growth factor comprises plasma or platelet-rich plasma;
(b) cell free fat extract or at least one growth factor comprises cell free fat extract;
(c) cell free stem cell extract or at least one growth factor comprises cell free stem cell extract;
(d) Extracellular Vehicles (EVs), secreted by stem cells;
(e) one or more essential amino acids;
(f) polynucleotide(PN) and/or polydeoxyribonucleotide (PDRN) extracted from the sperm cells of Oncorhynchus mykiss (Salmon trout) or Oncorhynchus keta (Chum Salmon) with a molecular weight ranging from 50 to 1500 kDa;
(g) local anesthesia drugs;
(h) stabilizer or dissolution promotor; and
(i) any combinations thereof.

3. The composition of claim 2, wherein
the concentration of active ingredient (a) is 1%˜50% by weight; and/or
the concentration of active ingredient (b) is 1%˜5% by weight; and/or
the concentration of active ingredient (c) is 0.1%˜5% by weight; and/or
the concentration of active ingredient (d) is 0.1%˜5% by weight; and/or
the concentration of active ingredient (e) is 0.1%˜5% by weight; and/or
the concentration of active ingredient (f) is 0.1˜2% by weight; and/or
the concentration of active ingredient (g) is 0.1% to 0.5% by weight; and/or
the concentration of active ingredient (h) is 0.1% to 5% by weight; and/or
active ingredient (g) is lidocaine or procaine; and/or
active ingredient (h) is Methyl sulfonyl methane (MSM).

4. The composition of claim 1, wherein
(a) the ratio of glycosaminoglycan to the neutral pH soluble collagen is between 10:1 to 1:10; or
(b) the concentration of glycosaminoglycan is in a range between 5 to 50 mg/ml.

5. The composition of claim 1, wherein the source of collagen is selected from allogeneic tissue, mammal tissue or marine species or axolotl hides derived matrix; and/or
the collagen is selected from full collagen or atelocollagen, or recombinant collagen or recombinant collagen peptides from microorganism, plants, insect cells or animal cells, or collagen mimic peptides.

6. The composition of claim 2, wherein the derivatized collagen is derivatized with acetylation agents that alter the pKa of collagen and has one or more of the following features:
(a) soluble at neutral pH;
(b) does not undergo fibrillogenesis at physiological pH; and/or
(c) precipitates at acidic pH.

7. The composition of claim 6, wherein the pH in feature (a) is pH 6.5-7.5; and/or the pH in feature (c) is pH 3.5-5.5.

8. The composition of claim 6, wherein the pH in feature (c) is pH 4.0˜5.0.

9. The composition of claim 2, wherein the derivatized collagen is derivatized with one or more agents selected from the group consisting of glutaric anhydride, succinic anhydride, maleic anhydride, citric acid anhydride, oxalic acid anhydride and ethylenediamine tetraacetic anhydride.

10. The composition of claim 2, wherein the neutral pH soluble collagen forms rapidly polymerizing collagen gels; and
the rapidly polymerizing collagen gels comprises a neutralized solution comprising an acid soluble collagen, EDTA/EGTA and a polyol, and wherein the acid soluble collagen comprises collagen selected from the group consisting of Type I collagen, Type II collagen, Type III collagen and combinations thereof.

11. The composition of claim 10, wherein the acid soluble collagen in a concentration between 5 and 70 mg/ml; and/or
wherein said EDTA is disodium EDTA; and/or
wherein said EGTA is disodium EGTA; and/or
wherein said EDTA or EGTA is in a concentration between 10 and 50 mM; and/or
wherein said polyol is a sugar alcohol; and/or
wherein said polyol is in a concentration between 2.5% and 4% (w/v); and/or
wherein said rapidly polymerizing collagen gels further comprises a disaccharide, fructose, or combinations thereof; and/or
wherein said rapidly polymerizing collagen gel has an osmolality of 280-360 mmol/kg.

12. The composition of claim 11, wherein the sugar alcohol is D-mannitol.

13. The composition of claim 1, wherein the glycosaminoglycan is one or more selected from the group consisting of hyaluronic acid, heparosan, heparin, chondroitin sulfate, dermatan sulfate, keratan sulfate, and any combinations thereof.

14. The composition of claim 1, wherein the glycosaminoglycan is derived from allogenetic tissue, mammal tissue or marine species; and/or is produced through microbial fermentation.

15. The composition of claim 1, wherein the molecular weight of glycosaminoglycan before crosslinking is from 1000 Da-10000000 Da.

16. The composition of claim 2, wherein the crosslinker crosslinking glycosaminoglycan are independently selected from 1,4-butanediol diglycidyl ether (BDDE), 1-[3-(Dimethylamino)propyl]-3-ethylcarbodiimide methiodide (EDC), polyethyleneglycol diglycidyl ether (PEGDE), N,N′-dicyclohexylcarbodiimide (DCC), N,N′-diisopropylcarbodiimide (DIC), Diepoxyoctane (DEO), Divinyl Sulfone (DVS), glutaraldehyde, or p-phenylene biscarbodiimide or 1,2,7,8-diepoxyoctane, or Polyethylene glycol (PEG), or oligomers rich in amino groups or combinations thereof.

17. The composition of claim 16, wherein the oligomers rich in amino groups is selected from lysine, poly-lysine, poly-arginine or γ-polyglutamic acid.

18. The composition of claim 13, wherein hyaluronic acid is selected from oligo-hyaluronan, hyaluronic acid produced by microbial fermentation using Streptococcus species or Bacillus species, or allogeneic or animal tissues derived hyaluronic acid.

19. A method of preparing a composition comprising (i) neutral pH soluble collagen; (ii) glycosaminoglycan; and (iii) optionally, other active ingredients, said method comprises one or more step selected from:
(a) combining part (i) with part (ii) to form an injectable homogeneous gel; or
(b) adding ethanol precipitated part (ii) to a salt or pH precipitate of part (i) and adding part (iii) (if present) and re-solubilizing the combination by dialysis or diafiltration or ultrafiltration process to form a homogeneous injectable gel; or
(c) combining part (i), part (ii) and part (iii) (if present), and re-solubilizing the mixture of lyophilized part (i), part (ii) and part (iii) and dialyzing the combination to neutral pH form a homogeneous injectable gel.

20. The method of claim 19, wherein in step (a), part (i) is combined with part (ii) by adding part (ii) to part (i) by utilizing vacuum planetary mixer to form an injectable homogeneous gel; and/or
wherein in step (a), part (i) is combined with part (ii) with a revolution speed of 200 rpm-1,400 rpm and an autorotation speed of 100 rpm˜700 rpm, and with a mixing time of 10˜30 minutes with vacuum under sterile condition; and/or
wherein in step (c), part (i), part (ii) and part (iii) (if present) are combined by sterile freeze-drying part (i) and part (ii) and part (iii).

21. A method for augmenting soft tissue or inducing a cellular growth promoting scaffold in a tissue space under an epidermis in a subject in need thereof, comprising administering the composition of claim 1 to a site in need of the augment or induction.

22. The method of claim 21, wherein the composition is injected into soft tissue to correct soft tissue deficiencies; and/or
wherein the composition is injected into dermis to correct soft tissue deficiencies including wrinkles, dermal folds, dermal laxity, unevenness, facial emaciation, fat atrophy, cheek depression, eye socket depression, or a combination thereof; and/or
wherein the composition is injected into tissues other than dermis, including cartilage, to correct tissue deficiencies; and/or
wherein the composition is injectable through a 25, 26, 27, 28, 29, 30 gauge needle or cannula.
